(19) United States
(12) Patent Application Publication
(10) Pub. No.: US 2010/0179364 A1
(43) Pub. Date: Jul. 15, 2010

(54) Process for the Production of a Hydrocarbon

(75) Inventors: Jay Alan Labinger, Claremont, CA (US); John Glenn Sunley, East Yorkshire (GB); Xingwei Li, Singapore (SG)

Correspondence Address: GREENLEE WINNER AND SULLIVAN P C, 4875 PEARL EAST CIRCLE, SUITE 200, BOULDER, CO, 80301, US

(73) Assignees: CALIFORNIA INSTITUTE OF TECHNOLOGY, Pasadena, CA (US); BP CHEMICALS LIMITED, Middlesex (GB)

(21) Appl. No.: 12/374,933

(22) PCT Filed: Aug. 23, 2007
(86) PCT No.: PCT/US07/76615

§ 371 Date: Nov. 23, 2009

Related U.S. Application Data
(60) Provisional application No. 60/839,709, filed on Aug. 24, 2006.

(51) Int. Cl. C07C 1/20

(52) U.S. Cl. 585/639; 585/733

(57) ABSTRACT

A process for the production of a hydrocarbon which comprises contacting, in a reactor, methanol and/or dimethyl ether with a catalyst comprising a metal halide, such as a zinc halide, in which the methanol and/or dimethyl ether is contacted with the catalyst in the presence of at least one phosphorus compound having at least one P—H bond.

### CROSS-REFERENCES TO RELATED APPLICATIONS

This application claims the benefit of priority to U.S. Provisional Patent Application No. 60/839,709, filed Aug. 24, 2006, which is hereby incorporated by reference in its entirety to the extent not inconsistent with the disclosure herein.

### STATEMENT ON FEDERALLY SPONSORED RESEARCH OR DEVELOPMENT

Not applicable.

### BACKGROUND OF THE INVENTION

This invention relates to a process for preparing hydrocarbons and in particular to a process for preparing hydrocarbons from methanol and/or dimethyl ether.

Hydrocarbons may be produced by homologation of methanol and/or dimethyl ether. For example, U.S. Pat. No. 4,059,626 describes a process for the production of triptane (2,2,3-trimethylbutane) comprising contacting methanol, dimethyl ether or mixtures thereof with zinc bromide. U.S. Pat. No. 4,059,627 describes a process for the production of triptane from methanol, dimethyl ether or mixtures thereof using zinc iodide. WO02070440 relates to a continuous or semi-continuous process for the production of triptane and/or triptene from methanol and/or dimethyl ether in which co-produced water is removed from the reactor as the reaction proceeds. WO05023733 relates to a process for the production of branched chain hydrocarbons which comprises reacting methanol and/or dimethyl ether with a catalyst comprising indium halide. WO06023516 relates to a process for the production of branched chain hydrocarbons which comprises reacting methanol and/or dimethyl ether with a catalyst comprising a metal halide selected from rhodium halide, iridium halide and combinations thereof.

Pearson in J.C.S. Chem. Comm. 1974 p 397 relates to conversion of methanol or trimethyl phosphate to hydrocarbons by heating in phosphorus pentoxide or polyphosphoric acid.

Kaeding et al in J Catal. 61, 155-164 (1980) relates to conversion of methanol to water and hydrocarbons over ZSM-5 zeolite modified with phosphorus compounds. U.S. Pat. No. 3,972,832 relates to phosphorus containing zeolites.

There remains a need for an alternative and/or improved process for production of hydrocarbons from methanol and/or dimethyl ether.

Thus, according to the present invention there is provided a process for the production of a hydrocarbon which process comprises contacting, in a reactor, methanol and/or dimethyl ether with a catalyst comprising a metal halide, such as zinc halide, in which the methanol and/or dimethyl ether is contacted with the catalyst in the presence of at least one phosphorus compound having at least one P—H bond.

### SUMMARY OF THE INVENTION

The present invention solves the technical problem defined above by the presence of a phosphorus compound having at least one P—H bond in the reaction of methanol and/or dimethyl ether in the presence of metal halide catalyst to produce a hydrocarbon. Useful metal halide catalysts in the present invention include transition metal halides and early p-block metal halides. In embodiments particularly useful for generating hydrocarbon products having a significant yield of highly branched alkanes, such as triptane (2,2,3-trimethylbutane) and/or or triptene (2,3,3-trimethylbut-1-ene), the metal halide catalyst is a zinc halide, such as ZnI₂, ZnBr₂ or a combination of these.

The at least one phosphorus compound having at least one P—H bond may be selected from the group consisting of hypophosphorous acid [this may be represented by the empirical formula H(H₂PO₂) or structural formula I and may also exist in a tautomeric form HP(OH)₂], phosphorous acid [this may be represented by the empirical formula H₂(HPO₃) or structural formula II and may also exist in a tautomeric form P(OH)₃] and mixtures thereof.

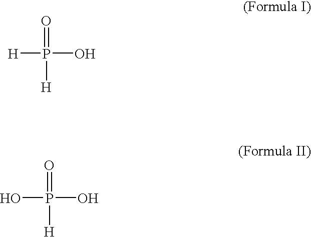

The at least one phosphorus compound having at least one P—H bond may be formed in situ by hydrolysis of one or more precursor phosphorus compounds in which the phosphorus is in a +3 oxidation state or lower. In the context of this description, the term “precursor phosphorus compound” refers broadly to compounds that generate at least one phosphorus compound having at least one P—H bond in the present methods, for example, via one or more chemical reactions. In some methods of the present invention, one or more precursor phosphorus compounds are provided that generate hypophosphorous acid, phosphorous acid or a combination of hypophosphorous acid and phosphorous acid via hydrolysis and/or other reactions such as decomposition reaction(s).

In some embodiments, precursor phosphorus compounds are one or more compounds having the empirical formulae: P(OR)₃, RP(OR)₂, R₂P(OR), HP(OR)₂, or H₂P(OR), wherein each R group is independently selected from the group consisting of H, an alkyl group, an alkenyl group and an aryl group. In a class of precursor phosphorus compounds useful in some methods of the present invention, each R group is independently selected from the group consisting of H and an alkyl group, and optionally the alkyl group has 1 to 4 carbon atoms, for example methyl, ethyl, n-propyl, iso-propyl. The R groups in each precursor phosphorus compound may be the same or different.

The process of the present invention is preferably performed with the at least one phosphorus compound having at least one P—H bond in the liquid phase.

Suitably, the at least one phosphorus compound having at least one P—H bond and/or its one or more precursors are present in the reactor in the process of the present invention at a concentration of 1 to 10 mol % relative to the methanol and/or dimethyl ether, and preferably for some applications the phosphorus compound and/or its one or more precursors are present in the reactor in the process of the present invention at a concentration of 5 to 10 mol % relative to the methanol and/or dimethyl ether. In the context of this description “mol %” refers to mole percentage, which in this description is 100 times the molar ratio of the phosphorus compound(s) having at least one P—H bond to the methanol and/or dimethyl ether.

Without wishing to be bound by any theory, it is believed that the at least one phosphorus compound having at least one P—H bond used in the present invention may be converted during the reaction, at least in part, to phosphoric acid. If the phosphorus compound is converted to phosphoric acid, such phosphoric acid, if formed, may be converted back to a phosphorus compound having at least one P—H bond and/or one or more precursors thereof either within the reactor or by removing the phosphoric acid from the reactor and converting it back to a phosphorus compound having at least one P—H bond and/or one or more precursors thereof.

Suitable conditions for the process of the present invention are described for example in International Publication Nos. WO02070440, WO05023733 and WO06023516 the contents of which are incorporated by reference. The present methods provide both continuous and semi-continuous processes for the production of hydrocarbons. Methods of the present invention may further comprise the step of heating the mixture of methanol and/or dimethyl ether, catalyst comprising a metal halide, and phosphorus compound(s) having at least one P—H bond. In some embodiments, for example, the process of the present invention is carried out at a temperature greater than 100 degrees Celsius. Preferably for some applications, the process of the present invention is carried out at a temperature selected over the range of 100 degrees Celsius to 450 degrees Celsius, and more preferably for some applications at a temperature selected over the range of 200 degrees Celsius to 350 degrees Celsius.

Catalysts useful in the present methods include, transition metal halide and early p-block metal halides having the formula: MB_{y} and combinations of these, wherein M is a metal selected from the group consisting of Zn, Mn, Fe, Ru, Co, Rh, Ir, Ni, Pd, Pt, Cu, Cd, Al, In, and Sn, and wherein B is a halogen selected from the group consisting of Cl, Br and I, and wherein y is the oxidation state of M. Metal halides of the present invention include, but are not limited to, ZnI₂, ZnBr₂, MnI₂, FeI₂, RuI₃, CoI₂, RhI₃, IrI₃, NiI₂, PdI₂, PtI₂, CuI, CdI₂, AlI₃, InI, InI₃, InBr₃, SnI₂, SnI₄, and combinations of these. Use of metal iodides and bromides is preferred for some methods of the present invention. In some embodiments wherein the metal halide is a metal chloride, such as InCl₃, the process of the present invention is preferably carried out at a temperature above room temperature, such as a temperature selected over the range of 200 degrees Celsius to 450 degrees Celsius. As will be understood by those having skill in the art, metal halide compounds useful in the present invention may be present in a solvated or dissolved form comprising one or more cations and ions, such as metal cations and halogen anions, may be present in the form of a metal salt, or may be present in both a solvated or dissolved form and in the form of a metal salt. The metal halide catalyst may be completely dissolved or may be provided in solid and dissolved states. The metal halide may be directly introduced into the reactor or may be formed in-situ by reaction of a metal source and halide source.

In an embodiment useful for the generation of highly branched alkanes, such as triptane (2,2,3-trimethylbutane) and/or or triptene (2,3,3-trimethylbut-1-ene), the metal halide of the present methods is selected from the group consisting of: zinc halide, iridium halide, rhodium halide, indium halide or any combinations of these. In an embodiment of this aspect of the present invention, the metal halide catalyst of the present methods is selected from the group consisting of: ZnI₂, ZnBr₂, ZnCl₂, InI₃, InBr₃, InCl₃, RhI₃, RhBr₃, RhCl₃, IrI₃, IrBr₃, IrCl₃ or any combinations of these. Use of a zinc halide, such as zinc iodide or zinc bromide or mixtures thereof, is preferred for some applications. A zinc halide preferred for some methods is zinc iodide.

A suitable salt of a metal halide, such as zinc halide, is preferably anhydrous but it may be used in the form of a solid hydrate. The molar ratio of methanol and/or dimethyl ether to metal halide, such as zinc halide, is optionally in the range 0.01:1 to 24:1, for example 0.01:1 to 4:1.

In some embodiments, selection of the composition of the metal halide provides a means of selectively adjusting the branching and product distribution(s) of the hydrocarbons generated using the present methods. Use of a zinc halide, such as ZnI₂ and/or ZnBr₂, for example, in some methods generates hydrocarbon products having a significant yield of highly branched alkanes, such as triptane (2,2,3-trimethylbutane) and/or or triptene (2,3,3-trimethylbut-1-ene). In other embodiments, use of an indium halide, such as InI₃, InBr₃ and/or InCl₃, in some methods generates hydrocarbon products having significant yields of smaller hydrocarbons, such as i-butane, 2-methylbutane, C₆ alkanes and C₅ alkanes.

The catalyst comprising metal halide, such as zinc halide, may be maintained in an active form and in an effective concentration in the reactor by recycling to the reactor, halide compounds, such as for example hydrogen iodide and/or methyl iodide from downstream product recovery stage(s), such as described in WO02070440.

In addition to methanol and/or dimethyl ether reactants, there may also be introduced to the reactor additional feedstock components. Suitable additional feedstock components include hydrocarbons, halogenated hydrocarbons and oxygenated hydrocarbons, especially olefins, dienes, alcohols and ethers. The additional feedstock components may be straight chain, branched chain or cyclic compounds (including heterocyclic compounds and aromatic compounds). In general, any additional feedstock component in the reactor may be incorporated in the products of the reaction. The methods of the present invention may further include the step of providing one or more additional feedstock components to the reactor.

Certain additional feedstock components may advantageously act as initiators for the reaction to produce branched chain hydrocarbons. In the context of the present description, the term “initiator” refers to an additive that causes a chemical reaction or series of chemical reactions to take place and/or enhances the rate of a chemical reaction or series of chemical reactions. In some embodiments, for example, an initiator causes a reaction to take place in the liquid phase that otherwise requires the presence of a solid phase or mixed phase. Suitable initiators are preferably one or more compounds having at least 2 carbon atoms selected from alcohols, ethers, olefins and dienes. Preferred initiator compounds are olefins, alcohols and ethers, preferably having 2 to 8 carbon atoms. Especially preferred initiator compounds are 2-methyl-2-butene, 2,4,4-trimethylpent-2-ene, ethanol, isopropanol and methyl tert-butyl ether. The methods of the present invention may further include the step of providing one or more initiators to the reactor.

In a further preferred embodiment, there is also present in the reactor one or more initiators selected from one or more of hydrogen halides and alkyl halides of 1 to 8 carbon atoms. Methyl halides and/or hydrogen halides are generally preferred. For the production of branched chain hydrocarbons from dimethyl ether (DME), methyl halides are especially preferred initiators. Preferably, the halide of the initiator is the same element as the halide of the zinc halide catalyst.

In some processes of the present invention, for example processes using an indium halide such as InI₃, InBr₃ and/or InCl₃, there is optionally also present in the reactor an initiator comprising one or more branched alkanes. Branched alkane initiators useful in specific embodiments include 2,3-dimethylbutane, 2,3-dimethylpentane, 2-methylbutane (iso-pentane) and 2-methylpropane (iso-butane).

There may also be introduced into the reactor hydrocarbons which stimulate the reaction, for example methyl substituted compounds, especially methyl substituted compounds selected from the group consisting of aliphatic cyclic compounds, aliphatic heterocyclic compounds, aromatic compounds, heteroaromatic compounds and mixtures thereof. In particular, such compounds may comprise methylbenzenes such as hexamethylbenzene and/or pentamethylbenzene.

In the process of the present invention isopropanol is preferably also present in the reactor.

The reaction product of the process of the present invention is a hydrocarbon, for example triptane (2,2,3-trimethylbutane) and/or or triptene (2,3,3-trimethylbut-1-ene). The aggregate of triptane and triptene products is referred to as triptyls. In an embodiment, the reaction product of the present methods is one or more C₆ alkanes, C₇ alkanes, and C₈ alkanes. In an embodiment, the reaction product of the present methods is one or more of xylene, trimethylbenzene, tetramethylbenzene, pentamethylbenzene, hexamethylbenzene, 2,4-dimethylpentane, 2-methylhexane, 3-methylhexane, and iso-butane. Reaction products of the present methods may be present in one or more liquid and/or vapor phases. In an embodiment, the reaction products of the present methods comprise first and second liquid phases, wherein the first liquid phase is a hydrophilic phase comprising water, methanol, dimethyl ether or any combinations of these, and wherein the second liquid phase is a hydrophobic phase comprising one or more hydrocarbons, such as, triptane and/or triptene.

Water produced in the process of the present invention is preferably removed from the reactor. An embodiment of the present invention further comprises the step of removing water from the reactor, for example by addition of a drying agent or by physical separation means.

The reaction of the present invention is usually performed at elevated pressure for example 5 to 100 barG, preferably 10 to 100 barG, more preferably at a pressure of 50 to 100 barG. Blends of hydrogen with gases inert to the reaction may be used to pressurise the reactor. A mixture of hydrogen and carbon monoxide may be used, such as described in WO02070440, the contents of which are incorporated by reference.

The process of the present invention may be performed as a batch or as a continuous process. When operated as a continuous process, reactants (methanol and/or dimethyl ether) may be introduced continuously, together or separately, into the reactor and the hydrocarbon product may be continuously removed from the reactor.

The hydrocarbon product may be removed from the reactor in a batch or continuous process together with zinc halide and water, these being separated from the hydrocarbon product and other products of the reaction, if present, and recycled to the reactor. Unreacted reactants may also be separated from the hydrocarbon product and recycled to the reactor.

The process of the present invention may be performed at a temperature in the range 100 to 450° C., preferably for some applications in the range 100-250° C.

As will be understood by those having skill in the art, a range of reactors may be used in the present methods. In an embodiment, for example, the process of the present invention is performed in a reactor which is suitably an adiabatic reactor or a reactor with heat-removal mechanism(s) such as cooling coils which may remove, for example, up to 20% of the heat of reaction.

### BRIEF DESCRIPTION OF THE DRAWINGS

FIG. 1 shows the ¹³C NMR spectrum of organic products obtained using the present methods with H₃PO₂ provided as an additive.

FIG. 2 shows the ¹³C NMR spectrum of the organic products obtained using the present methods without any phosphorus compound present.

FIG. 3 shows a GC trace of a typical reaction catalysed by InI₃.

FIG. 4 shows a MS of 2,3-dimethylbutane fraction from reaction between InI₃, ¹³C-labeled methanol and 2,3-dimethylbutane.

FIG. 5 shows a MS of triptane fraction from reaction between InI₃, ¹³C-labeled methanol and 2,3-dimethylbutane.

### DETAILED DESCRIPTION OF THE INVENTION

Referring to the drawings, like numerals indicate like elements and the same number appearing in more than one drawing refers to the same element. Unless defined otherwise, all technical and scientific terms used herein have the broadest meanings as commonly understood by one of ordinary skill in the art to which this invention pertains. In addition, hereinafter, the following definitions apply:

The term “phosphorus compound” refers to a compound containing at least one phosphorus atom. Phosphorus compounds having at least one P—H bond are useful in the present methods. Phosphorus compounds include, but are not limited to, hypophosphorous acid, phosphorous acid and mixtures thereof. Phosphorus compounds having at least one P—H bond may be provided and used directly in the present methods or, alternatively, may be generated in situ by chemical reactions, such as hydrolysis reactions, involving precursor phosphorus compounds.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. Cyclic alkyl groups include those having one or more rings. Cyclic alkyl groups include those having a 3-, 4-, 5-, 6-, 7-, 8-, 9- or 10-member carbon ring and particularly those having a 3-, 4-, 5-, 6-, or 7-member ring. The carbon rings in cyclic alkyl groups can also carry alkyl groups. Cyclic alkyl groups can include bicyclic and tricyclic alkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxyl group is an alkyl group linked to oxygen and can be represented by the formula R—O.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cyclic alkenyl groups include those having one or more rings. Cyclic alkenyl groups include those in which a double bond is in the ring or in an alkenyl group attached to a ring. Cyclic alkenyl groups include those having a 3-, 4-, 5-, 6-, 7-, 8-, 9- or 10-member carbon ring and particularly those having a 3-, 4-, 5-, 6- or 7-member ring. The carbon rings in cyclic alkenyl groups can also carry alkyl groups. Cyclic alkenyl groups can include bicyclic and tricyclic alkyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogens replaced with one or more fluorine atoms.

Aryl groups include groups having one or more 5- or 6-member aromatic or heteroaromatic rings. Aryl groups can contain one or more fused aromatic rings. Heteroaromatic rings can include one or more N, O, or S atoms in the ring. Heteroaromatic rings can include those with one, two or three N, those with one or two O, and those with one or two S, or combinations of one or two or three N, O or S. Aryl groups are optionally substituted. Substituted aryl groups include among others those which are substituted with alkyl or alkenyl groups, which groups in turn can be optionally substituted. Specific aryl groups include phenyl groups, biphenyl groups, pyridinyl groups, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms.

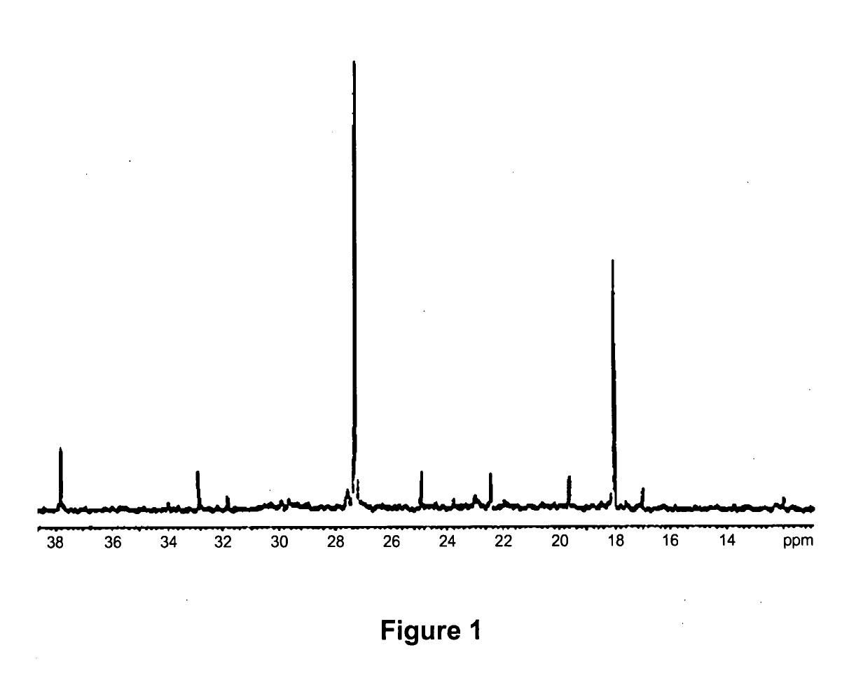

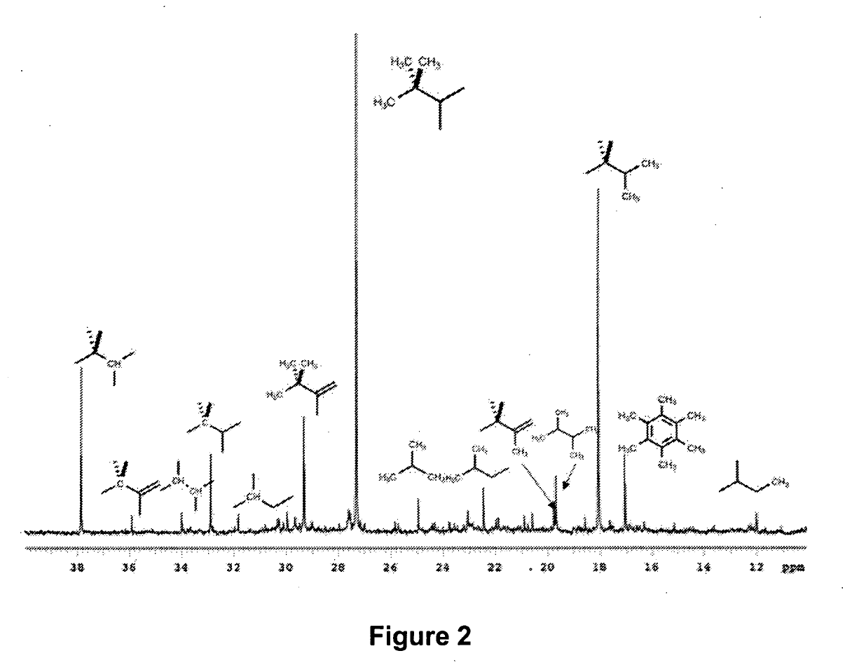

The invention will now be illustrated by way of example only and with reference to the following non-limiting examples and comparative experiments and with reference to FIGS. 1 and 2 which represent ¹³C NMR spectra of the organic products obtained using hypophosphorous acid and phosphorous acid, respectively.

### Example 1

All chemicals were purchased from Aldrich. Methanol was degassed but not dried prior to use. All other chemicals were used without any treatment.

To a thick-walled glass pressure tube (20 ml) was added zinc iodide (ZnI₂) (2.444 g, 7.65 mmol), methanol (1.0 ml, 791 mg, 24.7 mmol), isopropanol (50 μL, 39.2 mg, 0.65 mmol), and P(OCH₃)₃ (200 μL, 210 mg, 1.69 mmol) in this order under argon. This mixture was stirred to give a colorless or light yellow solution. The tube was then sealed and dipped into a preheated oil bath at 200° C., and was heated and stirred for 2 hours, after which time it was cooled to room temperature to give two layers. The top layer is colorless and the bottom one is orange with some precipitates. This mixture was chilled in ice water and a solution of cyclohexane in chloroform was added (1 ml, 83.4 mg cyclohexane in CHCl₃) and then water (1.0 ml).

The organic layer was extracted and analyzed by gas chromatography (GC) and found to contain 113 mg triptyls (triptene plus triptane) (average of two runs), which corresponds to a yield of 32% based on methanol, 26% based on total methyl groups and 24% based on all carbon atoms.

### Example 2

Example 1 was repeated using the same reaction condition but replacing the P(OCH₃)₃ with phosphorous acid H₃PO₃ (1.69 mmol).

86 mg for triptyls (triptene plus triptane) was obtained (averages of two runs) which corresponds to a yield of 24% based on methanol, 24% based on total methyl groups and 23% based on all carbon atoms.

### Examples 3 and 4

### Effect of Reaction Time

Reactions were also performed at different reaction time.

Example 3—for P(OCH₃)₃, under the same reaction conditions as Example 1 for a three-hour reaction, 108 mg for triptyls (triptene plus triptane) was obtained (average of two runs) corresponding to a yield of 31% based on methanol, 25% based on total methyl groups and 23% based on all carbon atoms. Example 4—for H₃PO₃, under the same reaction conditions as Example 1 for a three-hour reaction, 89 mg for triptyls was obtained (average of two runs) corresponding to a yield of 25% based on methanol, 25% based on total methyl groups, and 24% based on all carbon atoms.

### Example 5

### The Effect of Reaction Temperature

Example 5—for P(OCH₃)₃, under similar reaction conditions to Example 1 but at 175° C. for 24 hours, a clear colorless top layer and a black bottom layer were observed upon cooling to room temperature. A yield of 121 mg for triptyls was obtained (average of two runs) corresponding to a yield of 34% based on methanol, 28% based on total methyl groups and 26% based on all carbon atoms.

### Comparative Experiments A and B:

With no P(OCH₃)₃ or H₃PO₃ used, the yields of triptyls (triptene plus triptane) at different reaction times were also determined using the experimental procedure as in Example 1.

Experiment A—A two-hour reaction gave 43 mg triptyls (average of two runs), corresponding to a yield of 12% based on methanol and 11% based on the total carbon atoms.

Experiment B—A three-hour reaction gave 66 mg triptyls (average of two runs), corresponding to a yield of 19% based on methanol, and 18% based on the total carbon atoms. All these data are displayed in Table 1.

TABLE 1

| | | | | | | | Yield |
|---|---|---|---|---|---|---|---|
| | | | | | | Yield | based |
| | Phosphorus | | | | | based | upon |
| | compound | | | | Yield based | upon | total |
| | (mol. % relative to | Reaction | | Total | upon | methyl | carbon |
| | methanol | time | Temp. | triptyls | methanol | groups | atoms |
| | reactant) | (Hours) | (° C.) | (mg) | (%) | (%) | (%) |
| A | No additive | 2 | | 43 | 12 | 12 | 11 |
| B | No additive | 3 | | 66 | 19 | 19 | 18 |
| 1 | P(OCH₃)₃ (6.8%) | 2 | 200 | 113 | 32 | 26 | 24 |
| 2 | H₃PO₃ (6.8%) | 2 | 200 | 86 | 24 | 24 | 22 |
| 3 | P(OCH₃)₃ | 3 | 200 | 108 | 31 | 25 | 23 |
| 4 | H₃PO₃ | 3 | 200 | 89 | 25 | 25 | 23 |
| 5 | P(OCH₃)₃ | 24 | 175 | 121 | 34 | 28 | 26 |

Reactions were performed using different amounts of P(OCH₃)₃ and a procedure as in Example 1 using zinc iodide (ZnI₂) (2.444 g, 7.65 mmol), methanol (1.0 ml, 791 mg, 24.7 mmol), iso-propanol (50 μL, 39.2 mg, 0.65 mmol).

The reactions were performed at 200° C. for 2 hours. The results are given in Table 2.

TABLE 2

| | | | | Yield % |
|---|---|---|---|---|
| | P(OCH₃)₃ | Yield | Yield % | (based on |
| | (mol % relative to | triptyls | (based on | methyl |
| Example | methanol reactant) | (mg) | methanol) | groups) |
| 6 | 50 μL (1.7 mol %) | 65 | 18 | 17 |
| 7 | 75 μL (2.6 mol %) | 94 | 27 | 25 |
| 8 | 100 μL (3.4 mol %) | 100 | 28 | 25 |
| 9 | 200 μL (6.8 mol %) | 113 | 32 | 27 |
| 10 | 300 μL (10.2 mol %) | 102 | 29 | 22 |

### Examples 11, 13, 14, and 15

A reaction (Example 11) was performed using different amounts of isopropanol and a procedure as in Example 1 using zinc iodide (ZnI₂) (2.444 g, 7.65 mmol), methanol (1.0 ml, 791 mg, 24.7 mmol), iso-propanol (100 μL, 78.5 mg, 1.3 mmol) and P(OCH₃)₃ (200 μL, 210 mg, 1.69 mmol) at 200° C. for 2 hours. This gave 105 mg triptyls. This is about the same yield of triptyls obtained from the reaction with 50 μL isopropanol under the same conditions.

TABLE 3

| | | | | | | Yield |
|---|---|---|---|---|---|---|
| | Phosphorus | | | | Yield | based |
| | compound | | | | based | upon |
| Experi- | (mol. % | | Re- | | upon | total |
| ment/ | based upon | Iso- | action | Total | methyl | carbon |
| Ex- | methanol | propanol | time | triptyls | groups | atoms |
| ample | reactant) | μL | (Hours) | (mg) | (%) | (%) |
| A | — | 50 | 2 | 43 | 12 | 11 |
| B | — | 50 | 3 | 66 | 19 | 18 |
| 2 | H₃PO₃ | 50 | 2 | 86 | 24 | 22 |
| | (6.8%) | | | | | |
| 4 | H₃PO₃ | 50 | 3 | 89 | 25 | 23 |
| | (6.8%) | | | | | |
| 13 | P(OCH₃)₃ | 50 | 2 | 113 | 26 | 25 |
| | (6.8%) | | | | | |
| 14 | P(OCH₃)₃ | 50 | 3 | 108 | 25 | 24 |
| | (6.8%) | | | | | |
| 11 | P(OCH₃)₃ | 100 | 2 | 105 | 25 | 22 |
| | (6.8%) | | | | | |
| 15 | P(OC₂H₅)₃ | 50 | 2 | 67 | 19 | 13 |
| | (6.8%) | | | | | |

These experiments show the beneficial effect of the presence of a phosphorus compound having at least one P—H bond or a precursor thereof in the reaction of methanol and/or dimethyl ether in the presence of zinc halide catalyst to produce a hydrocarbon.

### Experiments Using Hypophosphorous Acid:

Hypophosphorous acid, H₃PO₂ is commercial available as an aqueous solution (50%) and the most stable tautomer is H₂P(O)OH.

In a typical experiment, an aqueous solution of hypophosphorous acid was charged into a thick-wall pressure vessel and was subjected to a vacuum for 12-36 hours, followed by the addition of zinc iodide (ZnI₂) (32 mol %), methanol (791 mg), and, if applicable, iso-propanol (i-PrOH). The reaction vessel was dipped into a preheated oil bath and was heated for a certain time, during which time white precipitates appeared. In most cases, the mixture was heated for 6-66 hours till the precipitates disappeared, depending on the loading of hypophosphorous acid and whether iso-propanol was used. In the cases where the amount of hypophosphorous acid was above 7.4 mol %, no dissolving of these precipitates had been observed and the reaction vessel was removed from the oil bath before the mixture turned light orange. The vessel was cooled to room temperature and the products were analyzed by GC or NMR.

As a comparison between H₃PO₃ and H₃PO₂ (7.4 mol %) (Examples 17 and 18), the reaction at 200° C. showed an increase of yield (based on total carbon) from to 26% for phosphorous acid (H₃PO₃) to 33% for hypophosphorous acid (H₃PO₂). The most striking differences between the reaction mixtures is that the triptane to triptene ratio is over 20:1 for the reaction using hypophosphorous acid based on both GC and ¹³C NMR analysis. FIG. 1 shows the ¹³C NMR spectrum of organic products obtained using the present methods with H₃PO₂ provided as an additive, and FIG. 2 shows the ¹³C NMR spectrum of the organic products obtained without any phosphorus compound present. The ¹³C NMR spectrum (FIG. 1) of the organic products with H₃PO₂ additive had a higher concentration of triptane than that without (FIG. 2) although hexamethylbenzene, isopentane, and 2,3-dimethylbutane are also present. The GC trace of the organic products also showed the presence of less aromatic compounds, including hexamethylbenzene (HMB).

Further experiments were performed using hypophosphorous acid and the results are shown in Table 4. Unless specified, the amount of zinc iodide was 32 mol %. The lowest effective amount of H₃PO₂ was 5.5 mol % and the presence of i-PrOH was necessary at this amount of H₃PO₂, but it proved not necessary for temperatures over 170° C. with an amount of hypophosphorous acid above 7.4 mol %. The maximum yields obtained at different temperatures are in the range of 33-37% (based on total carbon atom). Reproducibility of these reactions with respect to the reaction time is not as good as that with respect to the maximum yields, possibly due to the presence of different amounts of water and/or the nature of the heterogeneity of this reaction. Water in the H₃PO₂ solution slows down the reaction as shown in a reaction at 200° C. where no water in the aq H₃PO₃ solution was removed, but there is little effect on the ultimate yield. From Table 4, it is clear that reactions at lower temperatures tend to give higher yields and the maximum yield obtainable is about 37%.

TABLE 4
H₃PO₂ as additives at various conditions

| | | | | | | | Yield |
|---|---|---|---|---|---|---|---|
| | H₃PO₂ | | | | | | %, |
| | (mol. % | | | | | | based |
| | relative to | | | | | Yield %, | on total |
| | methanol | Temp. | Time | i-PrOH | Yield | based on | carbon |
| | reactant) | (° C.) | (hours) | (mol %) | (mg) | methanol | atoms |
| 17 | 7.4^{a} | 200 | 3 | 2.7 | 122 | 35 | 32 |
| 18 | 7.4^{a} | 200 | 5 | 2.7 | 127 | 36 | 33 |
| 19 | 7.4^{a} | 200 | 12 | 2.7 | 130 | 37 | 34 |
| 20 | 7.4^{a} | 200 | 6.3 | 0 | 116 | 33 | 33 |
| 21 | 7.4^{a} | 180 | 31.5 | 0 | 110 | 31 | 31 |
| | (25 mol % | | | | | | |
| | Znl₂) | | | | | | |
| 22 | 7.4^{a} | 180 | 26 | 2.7 | 115 | 33 | 31 |
| | (24 mol % | | | | | | |
| | Znl₂) | | | | | | |
| 23 | 7.4^{a} | 170 | 24 | 0 | 129 | 36.5 | 36.5 |
| | | 170 | 42 | 0 | 132 | 37 | 37 |
| 24 | 7.4^{a} | 160 | 26 | 2.7 | 128 | 36 | 33 |
| | | 160 | 30.5 | 2.7 | 128 | 36 | 32 |
| 25 | 7.4^{a} | 160 | 24 | 2.7 | 111 | 31 | 29 |
| | with 1% | | | | | | |
| | p-TSA^{d} | | | | | | |
| 26 | 8.8^{a} | 200 | 8 | 2.7 | 130 | 37 | 34 |
| 27 | 8.8^{a} | 200 | 8 | 0 | 117 | 33 | 33 |
| | | 200 | 10.5 | 0 | 111^{c} | 31 | 31 |
| 28 | 8.8^{a} | 180 | 18 | 0 | 117 | 33 | 33 |
| | | 180 | 24 | 0 | 126 | 36 | 36 |
| 29 | 8.8^{a} | 170 | 42 | 0 | 126 | 36 | 36 |
| 30 | 11.1^{a} | 200 | 8 | 2.7 | 133 | 38 | 35 |
| 31 | 5.5^{a} | 200 | 5 | 2.7 | 110^{c} | 31 | 29 |
| | | | 11 | 2.7 | 108^{c} | 30 | 28 |
| 32 | 5.5^{a} | 160 | 27 | 2.7 | 114 | 32 | 30 |
| | | | 30.5 | 2.7 | 130 | 37 | 34 |
| 33 | 5.5^{a} | 150 | 48 | 2.7 | 124 | 35 | 32 |
| | | 150 | 66 | 2.7 | 138 | 39 | 36 |
| 34 | 3.7^{a} | 180 | 19.5 | 2.7 | 92^{c} | 26 | 26 |
| 35 | 3.7% | 180 | 20.5 | 2.7 | 124 | 35 | 32 |
| | H₃PO₂ + | | | | | | |
| | 3.7% | | | | | | |
| | H₃PO₃ | | | | | | |

^{a}H₃PO₂ (50% aq) vacuumed over 12 hour,
^{b}H₃PO₂ solution used directly,
^{c}reactions run for too long with black solutions and a higher hexamethylbenzene to triptane ratio obtained,
^{d}p-TSA = para toluene sulphonic acid.

The ³¹P NMR studies indicated that H₃PO₂ was a stoichiometric reducing reagent. Three further experiments were performed (Examples 36 to 38). The first reaction with H₃PO₂ (8.8 mol %) and ZnI₂ (32 mol %) in methanol (1.0 mL) produced triptyls in 36% yield (180° C., 24 h). The volatile components were then removed under reduced pressure and the reaction vessel with residue was charged with methanol and methyl iodide (6.5 mol %). A yield of 27 mol % triptyls was obtained for the second experiment (180° C., 24 h). However, the yield decreased to 19% triptyls for a further reaction. The analysis of the aqueous solution by ³¹P NMR analysis revealed that H₃PO₄ was the only phosphorus species after the third experiment. Without being bound by any theory, this is consistent with the role of H₃PO₂ or H₃PO₃ as stoichiometric reducing reagents and it is ultimately oxidized to H₃PO₄.

These experiments show that H₃PO₂ is a most effective additive for the homologation of methanol to triptane. The reaction proceed with improved selectivity of triptane and the triptane to triptene ratio is over 20:1.

### Example 39

### Conversion of Methanol to Hydrocarbons Using Metal Halide Catalysts in the Presence of Phosphorus Compounds

To demonstrate the broad applicability of the present methods, the conversion of methanol to hydrocarbons, such as 2,2,3-trimethylbutane (common name: triptane), was studied for a range of metal halides, and in some cases, in the presence of an additive comprising a phosphorus containing compound. A number of metal halides were identified as providing significant yields of hydrocarbon products. In addition, the product branching of these reactions in the presence of phosphorus containing compounds was observed to vary significantly with the composition of the metal halide.

39.a. Conversion of Methanol Over Metal Halides

A number of iodide salts of the late transition and early p-block metals were screened using the standard conditions for ZnI₂-catalyzed dehydrative conversion of methanol into triptane: heating a mixture of methanol and the metal salt in a 3:1 molar ratio, along with a small amount of a initiator (10 mol % t-butyl methyl ether was used for these experiments) for 3 h at 200° C. in a closed thick glass vessel. Salts tested included MnI₂, FeI₂, RuI₃, CoI₂, RhI₃, IrI₃, NiI₂, PdI₂, PtI₂, CuI, CdI₂, AlI₃, InI, InI₃, SnI₂, and SnI₄. In all cases partial dehydration of methanol to dimethyl ether (DME) and formation of small amounts of methyl iodide were observed, and a number of the reactions produced some hydrocarbon products; but detectable levels of triptane were obtained only for three cases. Besides InI₃, RhI₃ and IrI₃ gave low yields of triptane (5±2% on the basis of moles carbon charged). In contrast triptane yields of up to 15±3% can be achieved using InI₃, comparable to the yield of triptyls (combined yield of triptane and triptene) obtained from reactions involving ZnI₂ (17±3%).

Production of branched alkanes, such as 2,2,3-trimethylbutane, from methanol and dimethyl ether in the presence of indium halide, rhodium halide and/or iridium halide is described in International Publication Nos. WO 2005/023733 and WO 2006/023516, which are hereby incorporated by reference to the extent not inconsistent with the present description.

Mixtures of InI₃ and methanol, in molar ratios varying from 1:2 to 1:4, along with a initiator (typically 2.5 mol % i-propanol) were heated in a closed vessel at 200° C. Approximately two hours are required for complete conversion of methanol/DME to hydrocarbons and water. Increasing the relative amount of methanol inhibits reaction: at a molar ratio of 1:5 only traces of triptane form under the above conditions. However, more than 5 equivalents of methanol per In can be converted as follows: 1-2 equivalents of methanol per In are added and the reaction is carried out as described, the reaction mixture is cooled and all volatiles removed in vacuo. A fresh charge of methanol is then added, and the cycle repeated. Using this protocol, activity for converting methanol to triptane appears to be sustained indefinitely. Analysis of the dried residue after a reaction cycle by powder-pattern XRD shows that InI₃ is the major species present.

Reactions can be carried out at temperatures as low as 160° C., although longer reaction times (about 8 h) are required to achieve complete conversion; no reaction is observed at 140° C. If DME is used as a feedstock the reaction proceeds more rapidly and at still lower temperatures: complete conversion is seen after 4 h at 160° C., and substantial formation of triptane is observed after 24 h at 120° C.; no reaction was found at 100° C. For comparison, ZnI₂ is inactive below 180° C. with methanol and 140° C. with DME.

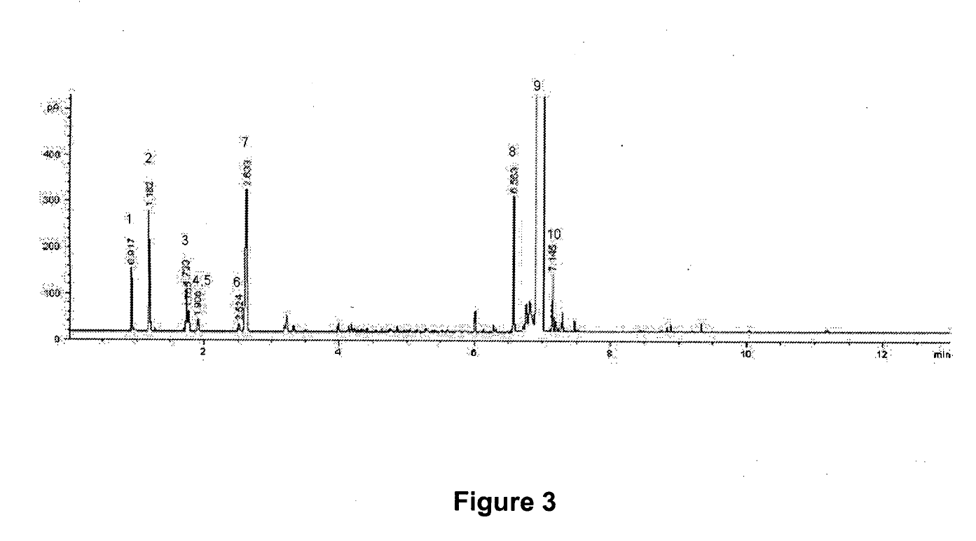

After cooling to room temperature, the reaction mixture contained two liquid phases (an upper organic layer and a lower aqueous layer) and a significant amount of solid. The organic layer was analyzed using a variety of techniques including GC, GC/MS, ¹H and ¹³C NMR spectroscopy. A typical GC trace is shown in FIG. 3. The largest peak in the GC trace is triptane; several other alkanes are present in significant quantities. The main arene peaks observed are pentamethylbenzene (PMB) and hexamethylbenzene (HMB). No methanol or dimethyl ether is observed in the organic layer.

Typical yields (determined by comparison of peak heights to that of an added internal standard, having previously calibrated response factors) are around 15% for triptane and 3% for HMB, based on total carbon in the feed (methanol plus initiator). As with ZnI₂, several factors must be controlled in order to obtain reproducible results. These include ensuring that the entire reaction vessel is heated so that there was no temperature gradient, only comparing results from vessels with the same headspace, and using reagents of the same purity.

Selected samples were subjected to PIANO (paraffin, iso-paraffin, arenes, naphthene, olefin) analysis, a standard refinery GC routine, which revealed that a large number of components were present. Selected results (including all major peaks) of the PIANO analysis are summarized in Table 5; results for an analogous reaction with ZnI₂ are included for purposes of comparison. The two major classes of compounds present are iso-paraffins and arenes, with a negligible amount of olefins.

TABLE 5
PIANO analysis results.

| Compound or Class | Weight %, InI₃^{a} | Weight %, ZnI₂^{a} |
|---|---|---|
| n-Paraffins | 0.6 | 1.3 |
| Isoparaffins | 58.7 | 45.0 |
| Arenes | 23.3 | 10.7 |
| Naphthenes | 4.6 | 5.2 |
| Olefins | 0.4 | 14.2 |
| i-butane | 2.8 | 2.6 |
| 2-methylbutane | 9.1 | 2.9 |
| 2-methylpentane | 2.3 | 0.4 |
| 3-methylpentane | 1.6 | 0.3 |
| 2,3-dimethylbutane | 5.3 | 1.8 |
| total C₆ isoparaffins | 9.1 | 2.5 |
| 2,3-dimethylpentane | 2.4 | 0.7 |
| 2,4-dimethylpentane | 1.5 | 0.4 |
| Triptane | 26.6 | 24.9 |
| total C₇ isoparaffins | 30.7 | 26.2 |
| Triptene | — | 5.6 |
| total C₈ isoparaffins | 4.3 | 3.8 |
| 1,2,3,5- | 1.7 | 0.5 |
| tetramethylbenzene | | |
| 1,2,4,5- | 1.2 | 0.3 |
| tetramethylbenzene | | |
| Pentamethylbenzene | 13.1 | 0.6 |
| Hexamethylbenzene | 5.5 | 3.4 |

^{a}As fraction of product organic layer.

Other indium halides are much less effective at generating triptane, as shown in Table 6: use of InBr₃ or InCl₃ as sole catalyst gives small amounts of or no triptane respectively, while even partially replacing InI₃ with either InBr₃ or InCl₃ reduces the yield of triptane.

TABLE 6
Effect of halide on triptane yield^{a}

| Molar % InI₃ | Molar % InBr₃ | Molar % InCl₃ | Triptane Yield (%) |
|---|---|---|---|
| 100 | 0 | 0 | 16.7 |
| 80 | 20 | 0 | 10.5 |
| 60 | 40 | 0 | 4.9 |
| 60 | 0 | 40 | 3.9 |
| 0 | 100 | 0 | 1.5 |
| 0 | 0 | 100 | 0 |

^{a}All reactions were performed using the standard reactions conditions (as described in Sections 39.a. and 39.c of Example 39) and with i-propanol added as an initiator. The combined molar ratio of MeOH:InX3 (X = I, Br, or Cl) was held fixed at 3:1.

In the absence of initiator, if the InI₃ is completely pre-dissolved prior to heating or stirred during heating, the solution remains homogeneous after 2 hours at 200° C., with no visible organic layer after cooling, and product analysis shows only the partial dehydration of methanol to DME. Initiator-free conversion can be still be achieved, so long as solid is present during the reaction. With additive, it makes no difference whether or not the mixture is pre-dissolved and/or stirred.

A number of additives may serve as initiators in addition to those mentioned above, including higher alcohols such as t-butanol and a wide variety of olefins ranging from terminal (1-hexene) to highly substituted (2,3-dimethyl-2-butene). Certain alkanes can promote conversion as well. Addition of 5 weight % of 2,3-dimethylbutane or 2,3-dimethylpentane gives results quite similar to those obtained with the initiators described above, except for significantly increased amounts of the alkane added as an initiator. Apparent recoveries of the latter (relative to amount added) are close to quantitative: 103% for 2,3-dimethylbutane and 91% for 2,3-dimethylpentane. However, since these alkanes are also products of methanol conversion, the values need to be corrected for the amounts formed in normal reactions, yielding values corresponding to 90% and 86% recovery, respectively. Several other alkanes, including triptane, 2,2-dimethylbutane, hexane and pentane fail to promote reaction in predissolved solutions of InI₃ in methanol: no new hydrocarbons form, only the partial dehydration of methanol to DME is observed, and the added alkane is recovered quantitatively.

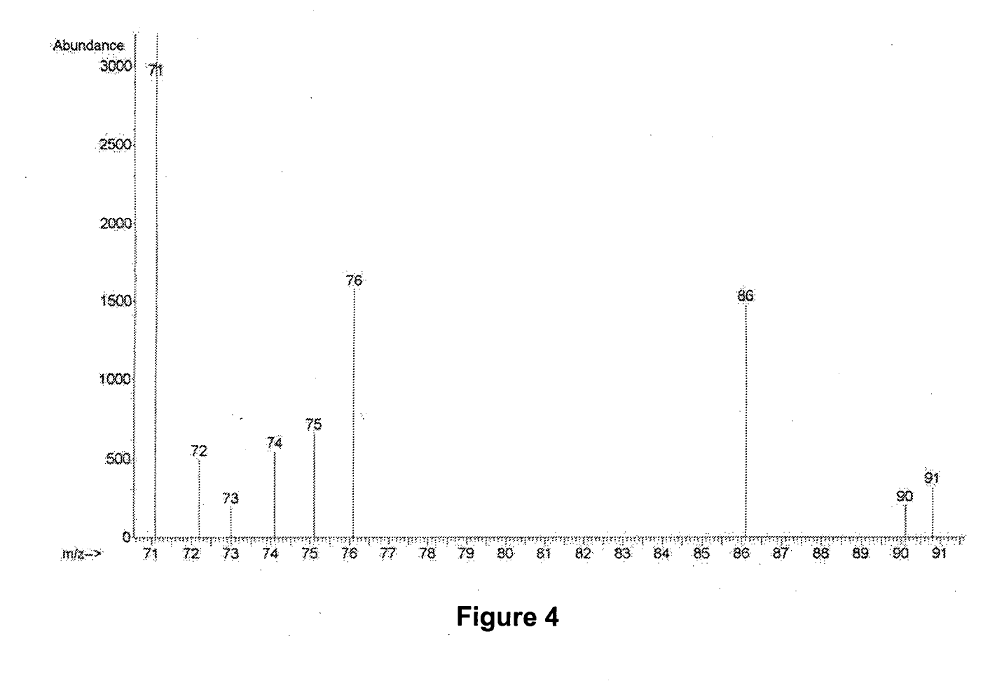

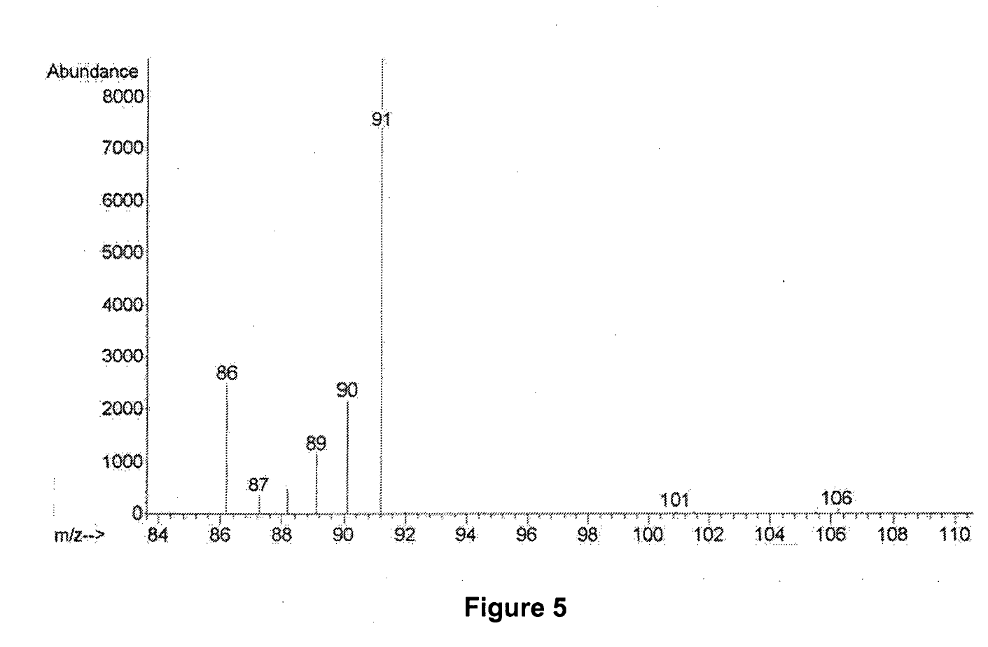

A similar experiment was carried out using 2,3-dimethylbutane as initiator and ¹³C-labeled methanol, both to verify that the alkane detected consists of both methanol-derived product and unreacted initiator, and to demonstrate the partial conversion of initiator to triptane. Products were analyzed by GC/MS; FIGS. 4 and 5 show the MS patterns for the GC fractions of 2,3-dimethylbutane and triptane, respectively. For the former, the major set of peaks from 71-76 m/z correspond to the (P-Me)⁺ fragment ions. Of these, the largest is at 71 (¹²C₅H₁₁) and the next-largest at 76 (¹³C₅H₁₁), with weaker peaks at intermediate values resulting from mixed isotopologs. There is also a P⁺ peak at 86 m/z for unlabeled 2,3-dimethylbutane, while the parent ions for other isotopologs are much weaker or not observed.

For triptane, the main signals again correspond to (P-Me)⁺ ions; there is barely any detectable signal in the P⁺ region. The largest signal at 91 m/z is due to fully labeled ¹³C₆H₁₃; the next largest, at 86 m/z, to singly labeled ¹²C₅¹³C₁H₁₃; weaker peaks are observed at intermediate values. However, there is no peak at 85 m/z, which would arise from completely unlabeled triptane.

The InI₃-catalyzed conversion of methanol to hydrocarbons exhibits many features quite analogous to those for catalysis by ZnI₂. In particular, reaction conditions are quite similar (although indium can be used at somewhat lower temperatures) and there are comparable yields of triptyls as well as hexamethylbenzene, a significant byproduct in both cases. Further parallels include the fact that hydrocarbon formation in the absence of a initiator can only be achieved if solid is present during the reaction. Additionally, in both systems conversion is significantly slowed or stopped altogether if the ratio of reactant (methanol or DME) to catalyst exceeds about 4:1, attributed to inhibition by water; when smaller amounts of reactant are converted over a single catalyst charge with removal of volatiles (including water) between runs, conversion can be continued indefinitely.

However, there are several major differences between the product distributions from the two catalyst systems, as shown in Table 5. Most notably, the yield of olefinic products from InI₃ catalyzed reactions is negligible, whereas around 14% of the products are olefins in the ZnI₂ system. In particular, only triptane is produced in the InI₃ systems, while both triptane and triptene are produced in ZnI₂ systems. In general the amount of iso-paraffins as well as arenes produced in indium reactions is considerably greater than in zinc reactions.

Differences within product classes between InI₃ and ZnI₂ catalyzed reactions can also be seen in Table 5. The selectivity for the maximally-branched alkane isomers is lower for InI₃ than for ZnI₂. In the ZnI₂ system the ratio of 2,3-dimethylbutane to other C₆ alkanes is around 3:1, while in the InI₃ system the ratio is approximately 5:4. A similar trend appears to be present for C₇ alkanes: the selectivity for triptane compared with other C₇ alkanes is not as high in InI₃ catalyzed reactions, although complete quantitative data could not be obtained for C₇ alkanes due to overlapping peaks in the GC trace. Another difference appears in the aromatic speciation: the ratio of hexamethylbenzene (HMB) to pentamethylbenzene (PMB) is much higher for zinc than indium.

39.b. The Effect of Phosphorus Reagents on InI₃ Catalyzed Reactions

Addition of H₃PO₃ or H₃PO₂ (6 mol % relative to methanol) substantially improves triptane yields in ZnI₂ catalyzed reactions. In contrast, addition of 6 mol % H₃PO₂ to reaction mixtures containing InI₃, MeOH and i-PrOH, results in a decreased yield of triptane, from approximately 15% to 10%, along with a significant increase in the yields of i-butane and 2-methylbutane, a smaller increase in the yield of C₆ alkanes, and a significant decrease in the yields of PMB and HMB (Table 7). ³¹P NMR spectroscopy shows that H₃PO₂ is oxidized to a mixture of H₃PO₃ and H₃PO₄ during the course of the reaction.

TABLE 7
Effect of 6 mol % H₃PO₂ on yield of selected species.

| | | % Yield from normal |
|---|---|---|
| | | reaction (i.e. without any |
| Compound | % Yield with H₃PO₂ | phosphorus additive) |
| i-butane | 10.7 | 7.5 |
| 2-methylbutane | 10.5 | 8.7 |
| 2,3-dimethylbutane | 2.2 | 2.9 |
| 2-methylpentane | 2.4 | 1.4 |
| 3-methylpentane | 1.5 | 0.9 |
| total C₆ isoparaffins | 6.1 | 5.2 |
| Triptane | 9.8 | 12.8 |
| Pentamethylbenzene | 3.6 | 7.1 |
| Hexamethylbenzene | 1.3 | 3.1 |

Without wishing to be bound by any theory, it is believed that the effect of addition of phosphorus reagents such as H₃PO₂ and H₃PO₃ to ZnI₂ catalyzed reactions are explained by the P—H bond containing species serving as alternate hydride sources, thus reducing the fraction of hydrocarbon that must be diverted from the triptane-producing sequence into the arene pool, resulting in an increase in the yield of triptane and a decrease in the yield of aromatic species. In contrast, addition of 6 mol % H₃PO₂ to InI₃ catalyzed reactions results in a decreased yield of triptane, accompanied by large increases in the yields of i-butane and 2-methylbutane and a smaller increase in the yield of C₆ alkanes. This suggests that when these phosphorus additives are used with InI₃, the rate of hydride transfer to carbocations is very fast relative to methylation of olefins; the conversion of lighter carbocations to alkanes becomes more efficient (compared to the Zn case) than carbon chain growth, and thus the selectivity for C₇ is reduced in favor of lighter alkanes. The observed decrease in the yields of PMB and HMB are consistent with hydrogen transfer from the phosphorus reagent, as is the observation (by ³¹P NMR spectroscopy) that H₃PO₂ is oxidized during the course of the reaction to a mixture of H₃PO₃ and H₃PO₄.

39.c. Experimental Section

Indium iodide, zinc iodide, methanol, dimethyl ether and other organic compounds were reagent-grade commercial samples used without further purification. ¹H, ¹³C and ³¹P NMR spectra were obtained on a Varian 300 MHz instrument. GC analyses were performed on an HP model 6890N chromatograph equipped with a 10 m×0.10 mm×0.40 μm DB-1 column. GC/MS analyses were performed on an HP model 6890N chromatograph equipped with a 30 m×25 mm×0.40 μm HP5-1 column and equipped with an HP 5973 mass selective EI detector.

The following metal salts were screened as potential catalysts for the conversion of methanol into triptane: MnI₂, FeI₂, RuI₃, CoI₂, RhI₃, IrI₃, NiI₂, PdI₂, PtI₂, Cu_{I}, CdI₂, AlI₃, GaI₃, InI, InI₃, SnI₂, and SnI₄. In all cases they were tested using the standard protocol described herein for InI₃ both in the presence and absence of an initiator. Only the InI₃, RhI₃ and IrI₃ systems showed any activity for the formation of triptane, although other metal halides led to the formation of other hydrocarbon products.

All reactions were performed in thick-walled pressure tubes equipped with Teflon stopcocks (Ace Glassware), rated up to 10 bar. The procedure for reactions involving InI3 is based on the procedure reported earlier for ZnI₂. In a typical experiment, the tube was equipped with a stir bar and charged with indium iodide (2.05 g, 4.1 mmol), methanol (0.5 mL, 12.4 mmol) and ⁱPrOH (50 μL) as an initiator. (The indium iodide was generally weighed out in a glove box due to its hygroscopic nature; however the reactions were carried out in air). The pressure tube was placed in a pre-heated oil bath behind a blast shield and stirred at 200° C. for the desired period of time, usually 2-3 hours. After heating, the tube was removed from the bath and allowed to cool to room temperature. The stopcock was removed and chloroform (1.0 mL), containing a known amount of cyclohexane as an internal standard, was pipetted into the reaction mixture followed by water (0.5 mL). The stopcock was replaced, the mixture was shaken vigorously and the organic layer separated. A small aliquot was diluted with acetone or tetradecane for GC analysis. In cases of samples to be used for NMR analysis, deuterated chloroform was used for the extraction.

In reactions involving dimethyl ether, all ingredients except DME were loaded into the tube. The tube was then degassed using three consecutive freeze-pump-thaw cycles and frozen in liquid nitrogen. The desired amount of DME was condensed into tube, which was allowed to warm to room temperature and then heated as usual.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

When a group of substituents is disclosed herein, it is understood that all individual members of those groups and all subgroups, including any isomers and enantiomers of the group members, and classes of compounds that can be formed using the substituents are disclosed separately. When a Markush group or other grouping is used herein, all individual members of the group and all combinations and subcombinations possible of the group are intended to be individually included in the disclosure. When a compound is described herein such that a particular isomer or enantiomer of the compound is not specified, for example, in a formula or in a chemical name, that description is intended to include each isomer and enantiomer of the compound described individually or in any combination. When an atom is described herein, including in a composition, any isotope of such atom is intended to be included. Specific names of compounds are intended to be exemplary, as it is known that one of ordinary skill in the art can name the same compounds differently. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example or illustration and not of limitation.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

We claim:

1. A process for the production of a hydrocarbon comprising contacting, in a reactor, methanol, dimethyl ether or both with a catalyst comprising metal halide, wherein the methanol, dimethyl ether or both is contacted with the catalyst in the presence of at least one phosphorus compound having at least one P—H bond.

2. The process of claim 1 wherein the at least one phosphorus compound having at least one P—H bond is selected from the group consisting of hypophosphorous acid, phosphorous acid and mixtures thereof.

3. The process of claim 1 wherein the at least one phosphorus compound having at least one P—H bond is provided in at a concentration of 1 to 10 mol % relative to the amount of methanol, dimethyl ether or both.

4. The process of claim 1 wherein the at least one phosphorus compound having at least one P—H bond is provided at a concentration of 5 to 10 mol % relative to the amount of methanol, dimethyl ether or both.

5. The process of claim 1 wherein the at least one phosphorus compound having at least one P—H bond is formed in situ by hydrolysis of one or more precursor phosphorus compounds, wherein the phosphorus in the precursor phosphorus compounds is in a +3 oxidation state or lower.

6. The process of claim 5 wherein the one or more precursor phosphorus compounds are one or more compounds having the empirical formulae: P(OR)₃, RP(OR)₂, R₂P(OR), HP(OR)₂, or H₂P(OR), wherein each R is independently selected from the group consisting of H, an alkyl group, an alkenyl group, and an aryl group.

7. The process of claim 6 wherein each R is independently H or an alkyl group having 1 to 4 carbon atoms.

8. The process of claim 1 wherein the metal halide is one or more compounds having the formula: MB_{y}, wherein M is a metal selected from the group consisting of Zn, Mn, Fe, Ru, Co, Rh, Ir, Ni, Pd, Pt, Cu, Cd, Al, In, and Sn; wherein B is a halogen selected from the group consisting of Cl, Br and I; and wherein y is the oxidation state of M.

9. The process of claim 8 wherein the metal halide is one or more compounds selected from the group consisting of ZnI₂, ZnBr₂, MnI₂, FeI₂, RuI₃, CoI₂, RhI₃, IrI₃, NiI₂, PdI₂, PtI₂, CuI CdI₂, AlI₃, InI, InI₃, InBr₃, SnI₂, and SnI₄.

10. The process of claim 1 wherein the metal halide is one or more compounds selected from the group consisting of: zinc halide, iridium halide, rhodium halide, and indium halide.

11. The process of claim 1 wherein the metal halide is one or more compounds selected from the group consisting of: ZnI₂, ZnBr₂, ZnCl₂, InI₃, InBr₃, InCl₃, RhI₃, RhBr₃, RhCl₃, IrI₃, IrBr₃, and IrCl₃.

12. The process of claim 1 wherein the metal halide is one or more zinc halide.

13. The process of claim 12 wherein the zinc halide is one or more compounds selected from the group consisting of ZnI₂ and ZnBr₂.

14. The process of claim 1 wherein the molar ratio of the methanol, dimethyl ether or both to the metal halide is selected over the range of 0.01:1 to 24:1.

15. The process of claim 1 wherein the hydrocarbon product comprises 2,2,3-trimethylbutane, 2,3,3-trimethylbut-1-ene or a combination of 2,2,3-trimethylbutane and 2,3,3-trimethylbut-1-ene.

16. The method of claim 1 further comprising the step of providing an initiator to said reactor.

17. The method of claim 16 wherein said initiator is one or more compounds having at least 2 carbon atoms selected from the group consisting of: alcohols, ethers, olefins and dienes.

18. A process for the production of a hydrocarbon comprising contacting, in a reactor, methanol, dimethyl ether or both with a catalyst comprising zinc halide, wherein the methanol, dimethyl ether or both is contacted with the catalyst in the presence of at least one phosphorus compound having at least one P—H bond.

19. The process of claim 18 wherein the at least one phosphorus compound having at least one P—H bond is selected from the group consisting of hypophosphorous acid, phosphorous acid and mixtures thereof.

20. The process of claim 18 wherein the at least one phosphorus compound having at least one P—H bond is formed in situ by hydrolysis of one or more precursor phosphorus compounds, wherein the phosphorus in the precursor phosphorus compounds is in a +3 oxidation state or lower.

21. The process of claim 18 wherein the zinc halide is one or more compounds selected from the group consisting of ZnI₂ and ZnBr₂.

22. The process of claim 18 wherein the hydrocarbon product comprises 2,2,3-trimethylbutane, 2,3,3-trimethylbut-1-ene or a combination of 2,2,3-trimethylbutane and 2,3,3-trimethylbut-1-ene.

23. The method of claim 18 further comprising the step of providing an initiator to said reactor.

24. The method of claim 23 wherein said initiator is one or more compounds having at least 2 carbon atoms selected from the group consisting of: alcohols, ethers, olefins and dienes.

25. The method of claim 23 wherein said initiator is one or more compounds selected from the group consisting of: 2-methyl-2-butene, 2,4,4-trimethylpent-2-ene, ethanol, isopropanol and methyl tert-butyl ether.
